(19) United States
(12) Patent Application Publication
(10) Pub. No.: US 2024/0158732 A1
(43) Pub. Date: May 16, 2024

(54) INCUBATION DEVICE AND MODULAR INCUBATION SYSTEM

(71) Applicant: ibidi GmbH, Gräfelfing (DE)

(72) Inventor: Johannes JÜRSCHICK, München (DE)

(21) Appl. No.: 18/509,186

(22) Filed: Nov. 14, 2023

(30) Foreign Application Priority Data
Nov. 14, 2022 (EP) 22207179.7

(51) Int. Cl. C12M 1/00

(52) U.S. Cl. CPC C12M 41/14; C12M 23/38; C12M 45/03

(57) ABSTRACT

The disclosure relates to an incubation device comprising a first incubation chamber, wherein a sample carrier holder and an examination device for examining a sample that is arranged in the sample carrier holder are arranged in the first incubation chamber, wherein the first incubation chamber has a connection opening for connecting to a second incubation chamber and an inlet opening for introducing the sample into the first incubation chamber, wherein a connection device is arranged at the connection opening, and wherein the connection device can be connected to a connection device which is arranged at a connection opening of a second incubation chamber. The disclosure additionally relates to a modular incubation system comprising the incubation device.

### CROSS REFERENCE TO RELATED APPLICATION

The present application claims priority to European Patent Application No. 22207179.7 filed on Nov. 14, 2022. The entire contents of the above-listed application are hereby incorporated by reference for all purposes.

### TECHNICAL FIELD

The disclosure relates to an incubation device and a modular incubation system.

### BACKGROUND

When examining living cells, environmental conditions close to in-vivo must be established and maintained in order for the cells to show physiologically relevant behavior and experimental results to be meaningful and reproducible. There are differences in the optimal conditions depending on the organ or type of cell, for example, in terms of temperature or the concentration of oxygen (O₂) or carbon dioxide (CO₂).

The cells are typically cultured in a cell medium that contains a specific concentration of salt and certain nutrients. If the cells are cultivated in an open system, then the cell medium evaporates at a relative humidity below 100%, which leads to a temporal dependence of the salt concentration. However, these are unphysiological environmental conditions for the cells. In order to be able to cultivate cells optimally instead, there should be an enclosed room around the cells that can be heated, gas-flushed, and humidified, so that constant and/or controllable conditions prevail over longer periods of time. A room that can be controlled in this way is called an incubation room or incubation chamber. Such incubation rooms are known from EP 2 148 921 A2.

If a new cell medium is now supplied to the cells or if the cells are to be subjected to a constant flow, this medium is typically supplied from a reservoir into the region of the cells by way of tubes. When this medium flows in tubing that traverses zones of different temperatures, then temperature differences or fluctuations in the concentration of dissolved gas in the cell medium are created which can result in the formation of unwanted bubbles that adversely affect the flow conditions. In addition, differences in relative humidity can cause condensation surfaces on which water that is dissolved in the air condenses.

To overcome these drawbacks, peripheral equipment can be provided for analyzing or for controlling incubated cell carriers in external incubation rooms. However, the disadvantage there is that two incubation systems have to be controlled and coordinated with one another, which can nevertheless produce different environmental conditions on a physiologically relevant level, and that tubes run in a non-incubated region between the incubation room and the peripheral equipment. In the case of small flows or amounts of liquid in particular, a drop in temperature, a change in the gas concentration, or the formation of bubbles can then occur within these tubes. In addition, the majority of the various peripheral equipment has different requirements and demand for space so that a complex system requiring a lot of room or space can result.

In light of the drawbacks mentioned, it is an object of the present disclosure to provide an incubation device and a modular incubation system that allow for greater flexibility. This object is satisfied by an incubation device and a modular incubation system as described herein.

### SUMMARY

According to the disclosure, an incubation device is provided comprising:
a first incubation chamber,
wherein the following are arranged in the first incubation chamber:
   a sample carrier holder, and
   an examination device for examining a sample which is arranged in the sample carrier holder,
wherein the first incubation chamber comprises a connection opening for connecting to a second incubation chamber and an inlet opening for introducing the sample into the first incubation chamber,
wherein a connection device is arranged at the connection opening, and
wherein the connection device can be connected to a connection device which is arranged at a connection opening of the second incubation chamber.

The incubation device according to the disclosure makes it possible to attach further incubation devices or incubation chambers by way of the connection device in order to construct an incubation system. Due to this modular structure, a user can flexibly create an incubation system according to his requirements which can comprise, for example, additional peripheral equipment. As a result, the incubation device exhibits greater flexibility. Typical peripheral equipment in the framework of the incubation device shall be described in more detail hereafter.

An incubation chamber has a connection opening at which a connection device is arranged, where the connection device is used to connect a further incubation chamber to the incubation device or the first incubation chamber, respectively. As a result, the incubation device has a modular character, it therefore represents a module for a modular incubation system, which shall be described hereafter. Furthermore, the incubation chamber has an inlet opening which can be used, for example, for the introduction of a sample into the sample carrier holder or for the removal from the sample carrier holder. Other objects can also be entered through the inlet opening into the first incubation chamber or removed from the first incubation chamber, or work can be carried out in the interior of the first incubation chamber.

The incubation device can also comprise a closure device. The closure device is used to close the connection opening. The closure device can be arranged directly at the connection opening or at the connection device. A closure with the closure device can in particular be gas-tight.

A closed incubation device, or incubation chamber, respectively, can be used as a stand-alone module for incubating a sample.

An incubation chamber is a room in which a temperature, a relative humidity, and a gas concentration, in particular of O₂ and CO₂, can be set or in particular be controlled in an open-loop manner. Such an incubation chamber is particularly suitable for optimally cultivating cells or other biological samples.

The examination device can be an image recording system with an optical component, for example, a microscope with an objective. The image recording system can also be the optical component alone.

The examination device can also be an impedance measurement system. It can be used to measure cell adhesion to surfaces using impedance changes between an electrode attached beneath the cells and a free electrode in a cell culture medium. Several analysis methods can also be combined in the present incubation device.

A sample to be examined can be arranged in a sample carrier mounted in the sample carrier holder. It can therefore be understood to be synonymous where examining a sample and examining a sample carrier is mentioned hereafter.

The connection device of the first chamber can be configured for a positive-fit and/or a force-fit connection to a second chamber.

The connection between the first incubation chamber and the second incubation chamber can be detachable in a nondestructive manner.

As a result, any removal of the second incubation chamber from the first incubation chamber can also be carried out with little effort. This is in particular a reversible connection so that a second incubation chamber can be mounted and removed several times.

The connection device can comprise a component of a screw connection, a bayonet lock, a magnetic connection with a positioning mechanism, or a hook-in connection, for example, with a toggle latch. Several of the said types of components can also be combined in the connection device.

The embodiments of the connection device mentioned are easy-to-use forms of connection for mechanical components. A second incubation chamber can therefore be attached to the incubation device with little effort. High flexibility of the incubation device is ensured in this way.

In other words, the coupling device of the first chamber disposes of an element or a component of the above-mentioned connection mechanisms. The connection device of a second incubation chamber, which is connected to the connection device of the first incubation chamber, has a corresponding mating member so that a complete screw connection, a bayonet connection, or a hook-in connection is established by connecting the two coupling devices.

In addition, there may be several ways or types of connecting the connection device. For example, a hook-in connection can be additionally secured with a screw connection. This allows for a more stable connection to be established between the first incubation chamber and the second incubation chamber.

The connection opening can have an area of at most 1 m², in particular at most 0.25 m².

In principle, the area of the connection opening can be selected according to the intended use of the incubation device. However, it should be ensured that gas exchange takes place between the first incubation chamber and a connected second incubation chamber so that the same conditions prevail in both incubation chambers. This exchange takes place without additional devices by way of diffusion, where the diffusion rate depends upon the area of the connection opening. In addition, the connection opening must have a larger area than any possible openings to an environment of the incubation device, since otherwise stable and isotropic environmental conditions cannot be ensured in the interior. Openings to the environment can be present, for example, to suction air from the environment into the incubation chamber in a controlled manner.

The incubation device can further comprise an interface in an outer wall of the first incubation chamber, where the interface disposes of a port for an electrical, gas-conveying, and/or liquid-conveying connection.

The interface can be connected to a corresponding interface of a second incubation chamber so that a closed line for gases and/or liquids (fluids) is established between the first incubation chamber and the second incubation chamber. The fluids can be used, for example, to supply a sample arranged in the sample carrier holder. By forming an interface, the lines in the first incubation chamber can be automatically connected when the second incubation chamber is mounted. It is then also not necessary, for example, to manually route the line through the connection openings of the two chambers This simplifies the construction of an incubation system and enables a compact design.

Electrical connections can be formed, for example, in the form of pressure-elastic pogo pins or spring-clamp contacts. An electrical device arranged in the incubation device, for example, pumps, electrically operated valves, or heating elements, can be connected by way of the electrical connection in the interface to a current and voltage supply that is connected outside at the interface. At the same time, a second incubation chamber can be connected to the incubation device, where the electrical connection of the interface of the incubation device and the electrical connection of the interface of the second incubation chamber are connected. In both cases, electrical connections in the interface of the incubation device contribute to greater flexibility of the incubation device described.

The interface can comprise in particular a port for transmitting open-loop and/or closed loop signals. Typical ports in this context are CAN bus, serial communication, I2C, and Ethernet. In the case of an incubation device described hereafter or an incubation system described hereafter with a closed-loop control device, closed-loop control signals are used to control active components of the incubation device or the incubation system in a closed-loop manner, for example, pumps or heating elements, on the basis of a corresponding measured value. The ports present in the interface for transmitting open-loop and/or closed loop signals facilitate the connection of the associated components of a control loop, enable a modular structure, and thereby increase the flexibility of the incubation device or an incubation system.

The interface can comprise a plug connection, in particular with a sealing element.

Plug connections are characterized by easy handling and use over a plurality of cycles. Therefore, the interface of the first incubation chamber can be easily and reversibly connected to an associated interface of the second incubation chamber, and the connection can be released again.

This plug connection can be configured, for example, in the form of a Luer or Luer lock connection. The interface of the first incubation chamber there comprises a female Luer or Luerlock adapter into which a corresponding mating member in the form of a male Luer or Luerlock adapter, which is formed at an interface of a second incubation chamber, can be inserted. A sealed connection can be established by using a sealing element. The plug connection, in particular a Luer or Luer lock connection, can be used in the context of liquids and/or gases and represents an easy-to-use and reliable mechanism.

A connection for gases and/or liquids can be present in particular between the interface of the first incubation chamber and the sample carrier holder or the sample in order to transport a gas or a liquid, that is passed at the interface into the first incubation chamber, to the sample which is arranged in the sample carrier holder. The connection can be established, for example, by way of a tube.

The first incubation chamber can comprise a second connection opening at which a second connection device is arranged.

This feature further increases the flexibility of the incubation device because a further incubation chamber can be attached in addition to a second incubation chamber. An incubation system constructed in this manner can comprise, for example, additional peripheral equipment and thereby provide additional possible cases of use.

The explanations previously given regarding the connection opening and the connection device arranged thereat can also apply in the same way to the second connection opening and the second connection device. However, this does not have to be the case and the first and the second connection opening, as well as the first and the second connection device, can be configured to be different.

The incubation device can additionally dispose of a second interface. In particular, the number of interfaces can correspond to the number of connection openings.

As described, the connection openings are used to connect a further incubation chamber to the incubation device. This entails that it can be advantageous to also provide an associated interface for connecting the second chamber. However, this does not necessarily have to be the case.

The incubation device can furthermore comprise a sample supply device for providing a gas or a liquid to the sample, where the sample supply device comprises in particular a pump with a liquid reservoir. The sample supply device can be arranged in particular in the interior of the incubation device.

A sample supply device designates a device that is configured to provide a fluid, in particular liquid nutrient medium with precisely defined properties to a biological sample. Said properties include, inter alia, the temperature and a gas concentration, in particular the concentration of O₂ and CO₂, in the nutrient medium. In addition, a sample supply device is able to transport the nutrient medium to a sample in the first incubation chamber.

A pump with a liquid reservoir furthermore represents a simple way of supplying a sample in the sample carrier holder with said liquid or said nutrient medium by pumping the nutrient medium through a connecting element, for example, a tube, to the sample.

The incubation device and in particular the connection device can be thermally insulated.

An incubation device or an incubation system is characterized in that stable and controlled conditions, in particular a constant temperature, prevail within the incubation chambers. Thermal insulation ensures that heat exchange between the interior of the incubation chamber and the environment is suppressed and the temperature inside the incubation chamber is particularly stable. The connection devices can be particularly susceptible to such heat exchange. Thermal insulation of the connection device therefore also contributes to the effect described.

The incubation device can furthermore comprise a heating element for adjusting a temperature in the first incubation chamber. Furthermore, the incubation device can comprise a chamber supply device for adjusting air humidity and/or a gas concentration, in particular an O₂ or CO₂ concentration in the first incubation chamber. The heating element and/or the chamber supply device can be arranged in particular within the first incubation chamber.

The incubation device can furthermore comprise an air diffuser, such as a fan.

The air diffuser accelerates the reduction of a gradient in temperature, gas concentration, or humidity within the incubation chamber so that equal conditions can be rapidly created in the interior of the incubation chamber.

The incubation system can further comprise a temperature sensor, air humidity sensor, and/or gas concentration sensor for determining at least one parameter of temperature, air humidity, and gas concentration in the first incubation chamber.

Several sensors of the same type, i.e., sensors that determine the same parameter can also comprised there. A sensor that can determine all of the parameters mentioned can also be comprised.

The sensors mentioned are used to monitor and control the conditions in the interior of the incubation chamber or of the incubation device. For example, the parameters determined or measured can be displayed to a user of the incubation device. In the event of a deviation in a parameter, the user can, if necessary, perform changes to the settings, in particular of the sample supply device, the chamber supply device, or the heating element, in order to adjust the parameters back to an intended value.

The incubation device can furthermore comprise a closed-loop control device which is configured to control said parameter in a closed loop manner based on an associated measured value from the sensor or sensors.

The term “closed-loop control” presently implies that this is a feedback-controlled closed-loop control device. The above-mentioned parameters, or at least one of them, are continuously adjusted by the closed-loop control device to a predetermined target value. For this purpose, the closed-loop control device receives measured values from the sensors in the form of an electronic signal from which the associated parameter value is determined. The closed-loop control device also comprises an open-loop control device with which the operation of the chamber supply device, the heating element, and the sample supply device can be controlled. In this way, the closed-loop control device influences the conditions in the interior of the incubation device and can control the parameters in an open-loop manner accordingly.

In order to implement a feedback loop, the closed-loop control device can also be given a target value for a specific parameter. This target value can be entered, for example, by a user. On the basis of this target value and the sensor signal, the closed-loop control device calculates an error signal that shows how the parameter at issue must be altered. The closed-loop control device accesses the supply devices of the incubation device accordingly to adjust the measured values to the target value. This operation can be performed simultaneously for several of the above-mentioned parameters. In addition, the closed-loop control process is carried out continuously.

One of the advantages of such a closed-loop control device is that it continuously controls the environmental conditions in a closed-loop manner in the Interior of the incubation device without a user having to actively intervene. In addition, the accuracy of adjusting a certain parameter by a closed-loop control device is higher than that by a human user, which is of great importance when cultivating a biological sample. In other words, a closed-loop control device allows for stable conditions over long periods of time and precise adjustment of the parameters to a predetermined target value.

The disclosure also provides a modular incubation system comprising:
an incubation device described above, and
a second incubation chamber with a connection opening,
wherein a connection device is arranged at the connection opening of the second incubation chamber,
wherein the connection device of the first incubation chamber is connected to the connection device of the second incubation chamber.

The incubation system fulfills the advantages in terms of increased flexibility as already discussed with regard to the incubation device. This is achieved, inter alia, by the modular structure of the incubation system.

The first incubation chamber and the second incubation chamber can be connected in a gas-tight manner.

As a result, the incubation system is better insulated from the environment because there is no gas exchange with the environment and the advantages mentioned in the context of the thermal insulation of the incubation device come into play.

The incubation system can furthermore comprise a sample supply device for supplying a gas or a liquid to the sample arranged in the sample carrier holder. The sample supply device can comprise in particular a pump with a liquid reservoir.

The sample supply device can be arranged in the second incubation chamber and be connected to the sample carrier holder or the sample by way of a connecting element, in particular a tube

The arrangement of the sample supply device in the second chamber emphasizes the modularity of the incubation system. Once a sample supply device is required for the incubation system, a corresponding second incubation chamber with a sample supply device arranged therein can be attached to the incubation device. The incubation system can therefore be adapted to individual needs. In addition, it is possible to construct a compact incubation system because not all components always need to be part of the incubation system or the incubation device.

The sample supply device can be connected to the sample carrier holder or the sample carrier. This connection can be formed to be direct. A tube, for example, can be used as a connection. In the case of a direct connection, it can be effected by the connection openings of the first and the second incubation chamber. For example, the tube is routed through the two connection openings.

An interface for connecting a gas-conveying and/or liquid-conveying connection can be arranged in an outer wall of the second incubation chamber, where the interface of the first incubation chamber is connected to the interface of the second incubation chamber so that a closed gas-conveying and/or liquid-conveying connection is given.

Another possible variant is that the outer wall of the first incubation chamber and/or the second incubation chamber is detachable or comprises detachable parts. For example, the first incubation chamber can be cuboid and one or more of the side walls or parts of the side walls can be detachable. This serves the purpose of attaching a further incubation chamber to the incubation device in place of the detachable part of the outer wall. The same connection device can be used there for attaching the second incubation chamber as for attaching the side wall. The detachable part of the outer wall can be configured such that, in the mounted state, it closes (in the case of a gas-conveying or liquid-conveying connection) or covers (in the case of an electrical connection) the interface in the outer wall of the incubation chamber. Once the detachable part has been detached, the interface is opened or uncovered, respectively, and can be connected to the associated interface of another incubation chamber.

In the case of a connection by way of the interface of the first incubation chamber and the second incubation chamber, a respective connection between the sample supply device and the interface of the second incubation chamber as well as between the interface of the first incubation chamber and the sample carrier holder or the sample carrier exists. If the case that the sample supply device is arranged in the second chamber, a connection, for example, in the form of a tube, can lead from the sample supply device to the interface of the second chamber. A connection exists there to the interface of the first chamber, which is in particular gas-tight and liquid-tight. A further connection leads from the interface of the first incubation chamber to the sample carrier holder or the sample carrier.

The nutrient medium provided by the sample supply device is intended to be provided at a sample that is arranged in a sample carrier. This sample carrier can be mounted in the sample carrier holder. For this purpose, the sample carrier holder can have appropriate ports or valves. When the sample carrier is inserted into the sample carrier holder, a liquid path opens from the sample carrier holder into the sample carrier so that the nutrient medium reaches the sample. Alternatively, it can be necessary for the connecting element to be connected directly from the sample supply device or the interface to the sample carrier once the latter has been mounted in the sample carrier holder.

Since the first incubation chamber and the second incubation chamber form a common incubated room, the same environmental conditions prevail in both incubation chambers. In particular, this means that no temperature gradients arise within the system. In the case of a liquid with a certain gas concentration, transport through a temperature gradient (a cold bridge) can lead to undesired bubble formation when the gas that is dissolved gasses out. The formation of bubbles can there have a negative impact on the flow conditions. This drawback is eliminated by the incubation system described for the reason that the nutrient medium is passed through an environment of constant temperature.

The incubation system can furthermore comprise a heating element which is configured to adjust a temperature in the first incubation chamber and/or the second incubation chamber. The incubation system can also comprise a chamber supply device that is configured to adjust a temperature, humidity, and/or gas concentration, in particular CO₂ and/or O₂ concentration, in the first incubation chamber and/or the second incubation chamber.

The heating element and/or the chamber supply device can be arranged in particular within the first incubation chamber and/or within the second incubation chamber.

External incubation chamber supply devices are typically provided in conventional incubation systems to control the temperature, humidity, and gas concentration in an open-loop manner in the interior of the system. Two advantages arise when providing a heating element and/or a chamber supply device within the incubation system. Firstly, the system can be formed to be more compact because there is no additional external equipment, but the heating element and/or the chamber supply device are already integrated into the system. Secondly, the environmental parameters provided by the heating element and/or the chamber supply device are transferred directly to the interior of the incubation system. An external device would first have to lead gases or liquids that are provided into the interior of the incubation chamber by way of a connection. Imperfect insulation of this connection can lead, for example, to a temperature change due to a cold bridge so that the settings at the external incubation chamber supply device do not correspond to the final conditions in the interior of the system. This disadvantage does not arise with the configuration presently described.

The heating element can be configured to adjust the temperature in the first incubation chamber and in the second incubation chamber independently of one another. Furthermore, the chamber supply device can be configured to adjust the air humidity and/or the gas concentration in the first incubation chamber and in the second incubation chamber independently of one another.

On the one hand, it can be an advantage to have the conditions in both incubation chambers not be the same. For example, long-term experiments can be carried out with the incubation device in which a nutrient medium is stored in the second incubation chamber before it is passed to the sample in the first incubation chamber. In this case, the culture medium should be stored at low temperatures to extend its shelf life. A low temperature means in particular a colder environment than when culturing cells. Conversely, the second incubation chamber can also be set to a higher temperature than the first incubation chamber. The advantage of this is that bubble formation in the nutrient medium in the cooler first incubation chamber is additionally reduced.

On the other hand, the two incubation chambers can also be set to the same parameters. In this case, the two incubation chambers are respectively controlled without any adjustment having to take place by diffusion. Accordingly, an equilibrium of the conditions in the interior of the incubation system is reached more rapidly.

The incubation system can furthermore comprise an air diffuser, for example, a fan, for exchanging a temperature, gas concentration, and/or humidity between the first incubation chamber and the second incubation chamber.

The air diffuser accelerates the reduction of a gradient in temperature, gas concentration, or humidity between the two incubation chambers so that the same conditions can be rapidly created in both incubation chambers. For example, this can be relevant where the exchange that occurs through diffusion through the supply opening is limited due to the size of the supply opening.

The first incubation chamber and/or the second incubation chamber can be thermally insulated from the environment. In particular, the connection devices of the first incubation chamber and the second incubation chamber can be thermally insulated from the environment.

The incubation system can furthermore comprise a temperature sensor, an air humidity sensor, or a gas concentration sensor for determining a parameter of temperature, air humidity and/or gas concentration in the first incubation chamber and/or the second incubation chamber.

With regard to the type of sensors, the same properties apply as were discussed in the context of the incubation device.

The sensors can there be attached at different points within the incubation system. For example, one or more sensors can be attached to the sample carrier holder or in its immediate vicinity for monitoring the conditions in a sample that is arranged in the sample carrier holder. At the same time, sensors can also be present in both incubation chambers, for example, to ensure that constant and isotropic conditions prevail within the incubation system. The arrangement of the sensors can apply to all types of sensors mentioned.

The incubation system can additionally comprise a closed-loop control device which is configured to control said parameter in a closed-loop manner based on an associated measured value of the parameter from the sensor or the sensors.

The closed-loop control device can be configured in the form of a central computing unit in the incubation device, where the closed-loop control device is in particular part of the incubation device. The closed-loop control device can be configured to control in a closed-loop manner the parameters to be controlled within the entire incubation system.

For this purpose, the closed-loop control device receives measured values from the sensors in the incubation chambers and is itself connected to the components of the incubation system to be controlled in an open-loop manner, for example, pumps or heating elements, in order to control their operation in an open-loop manner such that the parameters are adjusted to a predetermined target value. The connection between the closed-loop control device and the components to be controlled in an open-loop manner in adjacent incubation chambers can be established, for example, by way of the associated ports at the interfaces.

Alternatively, the closed-loop control device can be configured to be decentralized, where the closed-loop control process is carried out separately within each incubation chamber. For this purpose, each closed-loop control device receives target value for the parameters to be controlled in a closed-loop manner and controls the operation of active components in a closed-loop manner in the same incubation chamber based on sensor-measured values, where the associated sensors are also arranged in the same chamber as the corresponding closed-loop control device.

The incubation system can furthermore comprise a third incubation chamber, where the third incubation chamber comprises a connection opening with a connection device, where the first incubation chamber or the second incubation chamber comprise a second connection opening with a connection device arranged thereat, and where the connection device of the third incubation chamber is connected to the connection device of the second connection opening of the first incubation chamber or the second incubation chamber.

Depending on whether the third incubation chamber is attached to the first incubation chamber or the second incubation chamber, its arrangement is in parallel or in series with the first incubation chamber. The attachment of a third incubation chamber additionally increases the flexibility of the incubation system. For example, a chamber supply device can be arranged in the third incubation chamber, while a sample supply device can be arranged in the second chamber. The modular structure of the incubation system allows for more than two incubation chambers and therefore for a high level of flexibility in that an incubation system can be individually composed with specific devices. At the same time, it is possible to achieve a compact design of the incubation system. On the one hand, different peripheral equipment has individual space requirements to which the individual incubation chambers can be adapted. On the other hand, it is not necessary to always integrate all available equipment into one incubation system, as is often the case in conventional systems. Instead, only the required components can be mounted and integrated so that the incubation system takes up less space and is less complex.

In addition, the present disclosure provides a method for analyzing a sample in an incubation system or an incubation device, comprising
providing a previously described incubation device or a previously described incubation system;
inserting a sample carrier with a sample into the sample carrier holder; and
carrying out an examination on the sample carrier with the examination device.

The method in conjunction with the incubation system provides the advantages that the device also entails. They represent an examination of the sample in an incubation system that can be configured specifically for the examination. By arranging the sample supply device and possibly further devices within the incubation system, stable and optimal cultivation conditions can be provided for the sample.

A sample carrier is presently a stage for examining cells. In particular, the sample carrier can have a flat underside, which is particularly advantageous for microscopic examinations through this underside, especially in the case of inverse microscopy. Furthermore, the sample carrier is preferably made of transparent material, for example, plastic materials such as COC (cyclo-olefin copolymer), COP (cyclo-olefin polymer), PC (polycarbonate), PS (polystyrene), PE (polyethylene), PMMA (polymethyl methacrylate) or a transparent thermoplastic material or an elastomer or a mixture thereof.

Due to the use of the materials and methods mentioned, the sample carriers can be produced inexpensively and in large numbers having uniform quality. The reason for this is that injection molding with plastic materials is an established and reliable process and is particularly applicable in the case of the plastic materials mentioned. The use of transparent plastic material is particularly advantageous for being able to conduct optical examinations in the sample carrier, for example, by way of microscopy.

The sample carrier can comprise a channel and a reservoir, where the reservoir can be connected to the channel. For example, the sample is arranged in the reservoir and the nutrient medium flows through the channel and can thus supply the sample with nutrients.

The introduction of the sample carrier can also comprise that it is mounted in the sample carrier holder. For this purpose, the sample carrier holder can comprise a corresponding mount, such as a frame for the sample carrier, into which the sample carrier is ultimately screwed or otherwise affixed.

The method can comprise adjusting a parameter of temperature, humidity, and gas concentration in the first incubation chamber and/or the second incubation chamber while the measurement is being carried out. In particular, said parameter can be controlled in a closed-loop manner.

The statements provided regarding the advantages of controlling the parameters with an open-loop control device also presently apply. They comprise, inter alia, that the parameter is controlled in an open-loop manner without necessary supervision and can be set to be stable and precise over long periods of time.

The method described can furthermore comprise that a liquid in the second incubation chamber is heated at least temporarily to a higher temperature than in the first incubation chamber.

Since the examination device is arranged together with the sample carrier in the first incubation chamber, the liquid in a region of the examination is cooler than in the second incubation chamber. As a result, bubble formation in the liquid in the region of the examination can be additionally suppressed.

### BRIEF DESCRIPTION OF THE FIGURES

Further features and advantages shall be explained hereafter using the exemplary figures, where:

FIG. 1 shows a schematic incubation device as an oblique view;

FIGS. 2A to 2C show different schematic embodiments of a connection device of the incubation device;

FIG. 3A shows a schematic incubation system according to a first embodiment in cross section;

FIG. 3B shows a schematic incubation system according to a second embodiment in cross section;

FIG. 4 shows a schematic incubation system according to a further embodiment in cross section.

Hereinafter and in the figures, the same reference characters shall be used for the same or corresponding elements in the various embodiments, unless otherwise specified.

### DETAILED DESCRIPTION

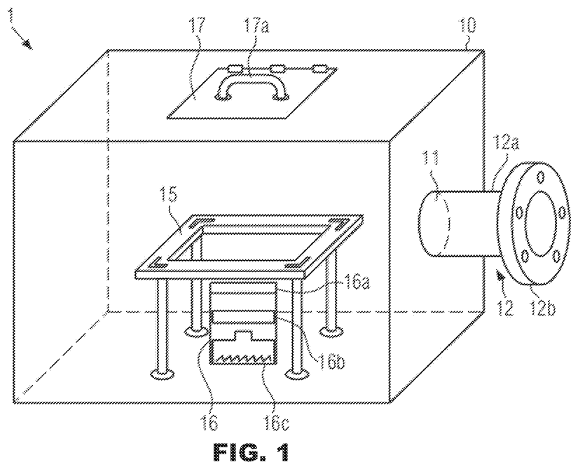

FIG. 1 shows an oblique view of an incubation device 1 according to a first embodiment. Incubation device 1 comprises a first incubation chamber 10. The outer wall of incubation chamber 10 can be made of, for example, glass or acrylic glass so that the outer wall is transparent. In principle, however, other materials that are common in the framework of incubation devices, such as metal sheets made of aluminum, white metals or stainless steel, insulation layers made of expanded polystyrene (Styrofoam), foam or other insulating materials, as well as injection-molded parts made of thermoplastics such as polycarbonate or polyvinyl chloride (PVC), can also be considered. The outer walls form the housing of incubation chamber 10.

An inlet opening 17 is formed in the upper side of the outer wall and in the case shown is configured in the form of a flap with hinges and a handle 17a. This is an example of an inlet opening 17 that is easy to implement and use. Inlet opening 17 can be used, for example, to introduce a sample into the interior of incubation device 1 or of first incubation chamber 10, respectively. Other objects can also be introduced into or removed from incubation chamber 10 or other work can be carried out in incubation chamber 10. For example, a tube can be laid within incubation chamber 10. Inlet opening 17 can also be attached at a different location of incubation device 1 and is also not restricted to the shape of a flap with a handle 17a.

Furthermore, incubation device 1 comprises a connection opening 11 which is formed at a different location on the outer wall than inlet opening 17. A connection device 12 is attached to connection opening 11 and is configured such that a second incubation chamber can be attached thereto which likewise comprises a connection opening with a connection device. Connection device 12 of first incubation chamber 10 and the second incubation chamber must match one another or be corresponding mating members such that a stable connection can be created therewith. In the example shown, connection device 12 comprises a pipe member 12a with a flange 12b. A second connection device of the second incubation chamber can likewise comprise a flange so that both connection devices can be flanged and screwed together.

In the event that incubation device 1 is to be closed, a blind flange can be attached to connection device 12. This blind flange corresponds to a previously described closure device.

Connection device 12 can basically be made of the same material as the outer wall of incubation chamber 10. However, it can also be advantageous to have connection device 12 be made of a different material than the outer wall of incubation chamber 10. For example, for a connection to a flange 12b, it can be advantageous to have flange 12b itself be made of metal, such as steel, or at least comprises it. When connected with metal screws, a flange 12b made of metal withstands a higher load than plastic or glass. This reduces the likelihood of damage to the incubation device due to high, possibly point-based, loads on coupling device 12.

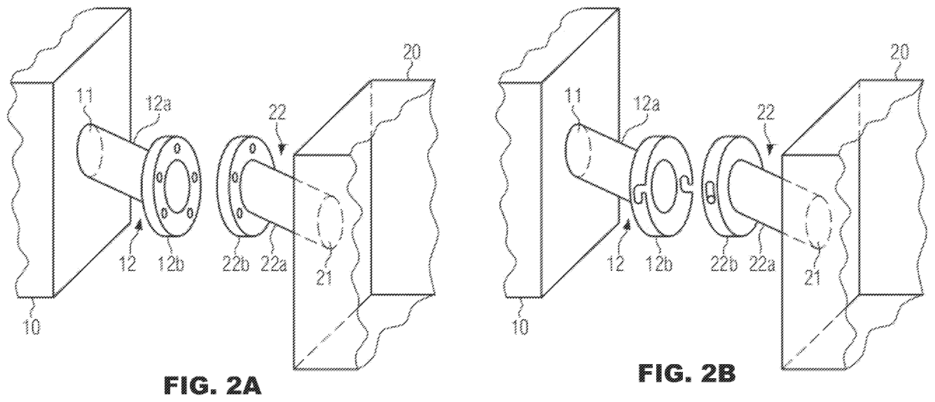

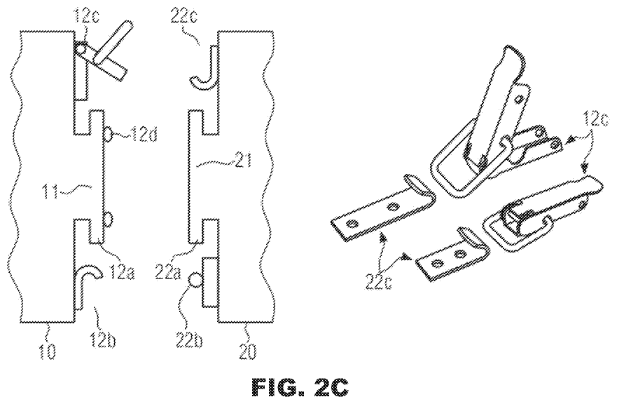

Connection device 12 is not restricted to the example shown and further examples shall be explained hereafter with reference to FIGS. 2A to 2C.

Incubation device 1 of FIG. 1 furthermore comprises a sample carrier holder 15 and an examination device 16 which are arranged in the interior of incubation chamber 10. In the present example, sample carrier holder 15 is configured in the form of a table on which a sample carrier can be placed and mounted. Disposed in the underside of sample carrier holder 15 is a recess in the table through which an examination with an examination device 16 can be carried out.

In the present case, examination device 16 is an optical device, or an image recording device, respectively, in the form of an inverted microscope. This microscope comprises a lens 16a, an eye member 16b and a camera 16c for recording images. The three components mentioned are surrounded by a tube to ensure greater stability and to prevent sources of interference such as scattered light from impinging on camera 16c. The image generated by camera 16c can be passed through an appropriate electronic connection from incubation chamber 10 to a processing device or a control interface (not shown), such as a computer.

In addition to the increased flexibility, another advantage of incubation device 1 described becomes apparent at this point. The optical components, in particular objective 16a of the microscope described, are disposed with the sample carrier and thus the sample in incubation chamber 10 of incubation device 1 and are therefore exposed to the same conditions. There is therefore no difference in temperature or humidity between the sample holder and objective 16a. This prevents liquid from condensing on objective 16a or the sample holder and falsifying the examination. This could possibly happen if objective 16a is in contact with the sample carrier and a temperature gradient between the sample carrier and objective 16a is given. This advantage also applies if examination device 16 is not an inverted microscope but rather a different device, for example, for measuring an impedance.

FIGS. 2A to 2C show different embodiments of connection devices 12, 22 with which first incubation chamber 10 of incubation device 1 and a second incubation chamber 20 can be connected to one another.

FIG. 2A shows a section of first incubation chamber 10 with connection opening 11 and a connection device 12 attached thereto. Starting out from connection opening 11, this connection device 12 comprises a pipe member 12a, at the end of which facing away from first incubation chamber 10 a flange 12b is attached. A second incubation chamber 20, which is to be connected to first incubation chamber 10, comprises a connection device 22 of identical structure with a pipe member 22a being attached to connection opening 21 and a flange 22b being attached to the former's end. The two flanges can be screwed together with screws or bolts (not shown) and connected in this manner.

In order to obtain improved sealing and thermal insulation of connected connection devices 12, 22 from the environment, a sealing element, for example, a sealing ring made of rubber or copper, can additionally be arranged between the two flanges. Such seals are used, for example, in the framework of vacuum devices.

Another form of a connection device 12, 22 shall be explained with reference to FIG. 2B. It differs from the connection device of FIG. 2A in that a bayonet lock is attached instead of a flange. Connection device 12 of first incubation chamber 10 comprises a pipe member 12a and, at its end facing away from incubation chamber 10, a rotatable outer member 12b with two oppositely disposed longitudinal slots, at the end of which a short transverse slot adjoins at a right angle. Connection device 22 of a second incubation chamber 20 comprises the associated mating member. Attached to connection opening 21 is a pipe member 22a, at the end of which an inner member 22b is arranged. Inner member 22b is pushed into outer member 12b and comprises a button that is guided into the longitudinal slot. By rotating outer member 12b, a firm connection is obtained in that the button latches into the transverse slot.

The arrangement of the connection device can also be reversed in the sense that connection device 12 of first incubation chamber 10 comprises the inner member and connection device 22 of the second incubation chamber 20 correspondingly comprises the outer member. Furthermore, a sealing element can also be used there. One advantage of a bayonet connection over a flange connection is that no tools and/or screws are required.

FIG. 2C shows a further embodiment of said connection devices in cross section. Connection device 12 of first incubation chamber 10 there first comprises a pipe member 12a at connection opening 11. Additionally disposed on the outer side of first incubation chamber 10, in which connection opening 11 is also arranged, is a hook 12b in the lower half and a toggle latch 12c in the upper half. Disposed on the facing outer side of second incubation chamber 20, in addition to a pipe member 22a which is attached to the connection opening 2, is a crossbar 22b in the lower half and a hook 22c in the upper half. When the connection devices of first incubation chamber 10 and second incubation chamber 20 are to be connected, crossbar 22b is first hooked into hook 12b on the outer side of first incubation chamber 10. Hook 22c is thereafter hooked into an eyelet of toggle latch 12c on the outer side of second incubation chamber 20 and toggle latch 12c is finally tightened. As a result, both pipe members 12a, 22a are pressed together and both incubation chambers 10, 20 are connected.

A detailed view of toggle latch 12c together with hook 22c is also shown in FIG. 2C. When connecting the connection device, hook 22c is hooked into the eyelet of toggle latch 12c as described and toggle latch 12c is finally tightened.

A sealing element 12d arranged between both pipe members 12a, 22a can further improve the tightness of the connection.

All three examples of a connection device shown have the common advantages of simple handling and reversibility. The connection can be established and released again several times without the need for spare parts because the connection can be released in a non-destructive manner.

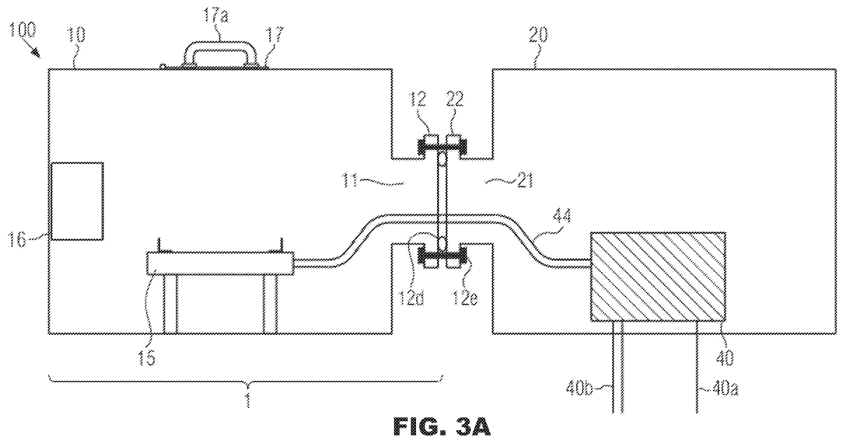

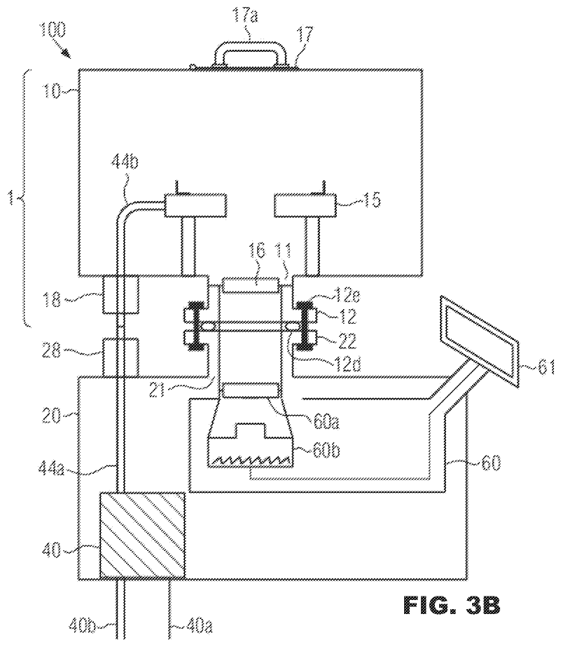

FIG. 3A shows an incubation system 100 according to a first embodiment as a schematic cross section. Incubation system 100 comprises an incubation device 1 with a first incubation chamber 10 as well as a second incubation chamber 20, where both incubation chambers are connected to one another by their connection devices 12, 22. Connection devices 12, 22 can, for example, but not necessarily, result in a flange connection, as explained in FIG. 2A. The flanges are there screwed with screws 12e and form in particular a gas-tight connection. A sealing element in the form of a sealing ring 12d is additionally arranged between coupling devices 12, 22 to improve the tightness and thermal insulation of the connection. Sealing ring 12d can be made, for example, of rubber or a different elastic material.

A sample carrier holder 15 and an examination device 16 are arranged in first incubation chamber 10. Examination device 16 is shown only schematically. For example, it can be an impedance measurement system. For this purpose, electrodes for measuring the impedance of a biological sample would have to be inserted into a sample carrier once the sample carrier is mounted in sample carrier holder 15. With regard to further details or embodiments of examination device 16, reference is made to the above explanations.

First incubation chamber 10 likewise comprises an inlet opening 17 with a handle 17a, as described above.

A sample supply device 40 is disposed inside second incubation chamber 20. It has a power supply 40a and an inlet 40b for gases and liquids. From the gases and liquids introduced, sample supply device 40 can produce a nutrient medium with a defined temperature and gas concentration. This nutrient medium is passed via a connection, such as a tube 44, to sample carrier holder 15 in first incubation chamber 10. Tube 44 leads through connection openings 11, 21 of first incubation chamber 10 and second incubation chamber 20. Valves can be present in sample carrier holder 15 so that the nutrient medium is delivered to a sample, so that the nutrient medium can flow into the sample carrier when a sample carrier is mounted. Alternatively, tube 44 can also be attached directly to the sample carrier after the sample carrier has been mounted.

It goes without saying that there must be a closed liquid circuit present for the nutrient medium. For this purpose, the nutrient medium is returned from sample carrier holder 15 to sample supply device 40. This can be accomplished by way of a second tube which is not shown in the figure.

An incubation system 100 comprising an incubation device 1 described and a second incubation chamber 20 according to a further embodiment is shown in FIG. 3B. The two incubation chambers are connected to one another by way of their respective connection devices 12, 22. As described in the above embodiments, the connection devices can in particular form a flange connection, where the two flanges are held together by screws 12e, and a sealing element 12d arranged between the flanges ensures better insulation of incubation system 100 from the environment.

Incubation device 1 first comprises a first incubation chamber 10 in which a sample carrier holder 15 and an examination device 16 in the form of an objective are arranged. In addition, incubation chamber 10 disposes of an inlet port 17 with a handle 17a on the upper side thereof and a connection port 11 on the underside thereof.

Second incubation chamber 20 in its interior comprises further components of a microscope 60, which is completed by the objective described. Said further components are an eye piece 60a, a camera 60b with a sensor for image generation, and a tripod (not shown). Outside second incubation chamber 20, microscope 60 can additionally dispose of a display device 61 with which an image generated by camera 60b is displayed to a user.

Furthermore, a sample supply device 40 is provided in second incubation chamber 20 and is configured to supply a gas and/or a liquid for a sample arranged in sample carrier holder 15. Chamber supply device 40 can comprise, for example, a pump which pumps a nutrient liquid at a specific temperature and/or a specific gas concentration to the sample. In the example shown, this is done with a line, such as a tube 44a, 44b, which is connected from sample supply device 40 via the connection of interfaces 18, 28 of first incubation chamber 10 and second incubation chamber 20 to sample carrier holder 15. With regards to the supply of a sample with the nutrient medium by sample supply device 40 and further properties of sample supply device 40, the same considerations apply as in the embodiment shown with reference to FIG. 3A In particular, sample supply device 40 comprises a power supply 40a and an input 40b. For example, liquids or other components for providing the nutrient solution can be supplied via this inlet 40b.

In addition to connection devices 12, 22, interfaces 18, 28 of first incubation chamber 10 and of second incubation chamber 20 are also connected to one another establishing a closed liquid path via tubes 44a and 44b from sample supply device 40 to sample carrier holder 15. Both sections of tube 44a, 44b are connected to one another at interface 28 in a liquid-tight manner, for example, in the form of a Luer lock connection.

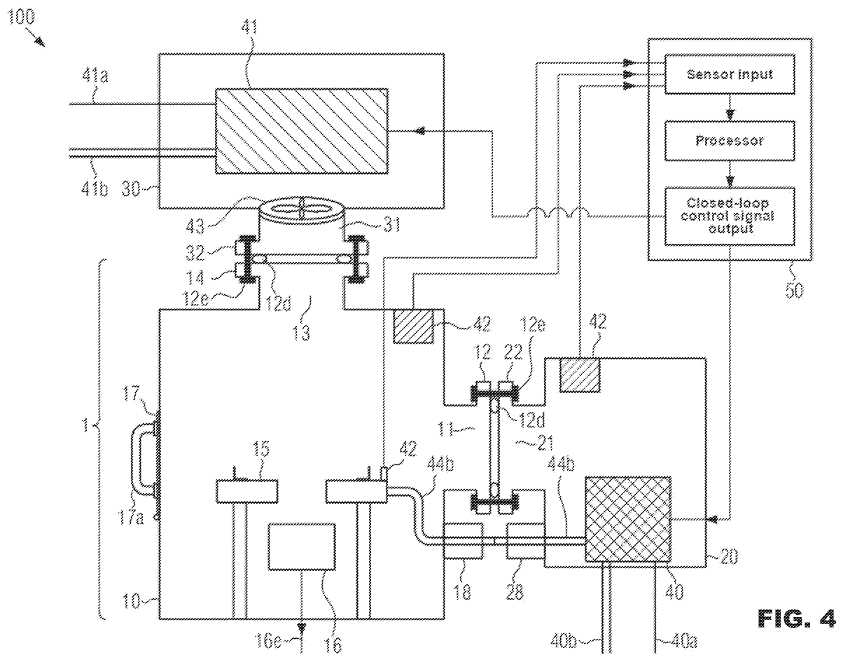

FIG. 4 shows an incubation system 100 comprising an incubation device 1 and two further incubation chambers, i.e. a total of three incubation chambers. As in the previous embodiments, an examination device 16 and a sample carrier holder 15 are also arranged in first incubation chamber 10, where examination device 16 is shown only schematically there. This can be, for example, an image recording system or impedance measurement system described. Measurement results from the examination device can be transmitted from the chamber via line 16e, for example, to an analysis device (not shown). An inlet opening 17 in the form of a flap and with a handle 17a is attached laterally to first incubation chamber 10.

Incubation system 100 also comprises a second incubation chamber 20, connection device 22 of which is connected to connection device 12 of first incubation chamber 10. These connection devices 12, 22 can be formed in the form of flanges and screwed together using screws 12e or bolts. Overall, connection devices 12, 22 are only shown in simplified form and are not restricted to a screw connection with flanges. Reference is made to the details above.

A sample supply device 40 is arranged in the interior of second incubation chamber 20. It has a liquid inlet 40b and a power supply 40a. The connections leading to these inlets can be present from the outside through passage openings in an outer wall of second incubation chamber 20. An outlet of sample supply device 40 is configured in the form of a tube 44a which can deliver a nutrient medium with the temperature and gas concentration provided to sample supply device 40. This tube 44a leads to an interface 28 of second incubation chamber 20.

First incubation chamber 10 likewise disposes of an interface 18 which is connected to interface 28 of second incubation chamber 20. This creates a closed liquid path for said nutrient medium from second incubation chamber 20 into first incubation chamber 10 in that tube 44a is connected to tube 44b at interfaces 18 and 28, for example, by way of a Luerlock connection. In first incubation chamber 10, tube 44b connects interface 18 to sample carrier holder 15. Sample carrier holder 15 can dispose of a valve so that the nutrient medium can flow from the sample carrier into an inserted sample carrier. Alternatively, the tube can also be connected directly to a sample carrier once it is mounted in sample carrier holder 15.

In addition, incubation system 100 furthermore has a third incubation chamber 30, where second connection device 14 of first incubation chamber 10 is connected to a connection device 32 of third incubation chamber 30. The interiors of first incubation chamber 10 and third incubation chamber 30 are thereby connected by connection openings 11 and 31. This connection can be configured in the same manner as between first incubation chamber 10 and second incubation chamber 20, but can also be configured in a different way. A chamber supply device 41 is arranged in third incubation chamber 30. It is primarily configured to adjust the air humidity and the gas concentration within incubation system 100. For this purpose, incubation chamber supply device 41 has a power supply 41a and a water connection 41b as inputs which are led into third incubation chamber 30 from the outside through passage openings provided. Chamber supply device 41 can also dispose of a heating element for being able to heat air accordingly. Overall, chamber supply device 41 is configured to generate air at a certain temperature and humidity and to deliver it to the interior of third incubation chamber 30.

An air diffuser, in particular a fan 43, is also arranged in third incubation chamber 30. In this case, fan 43 is attached to connection opening 31 of third incubation chamber 30. Fan 43 distributes the air from the third incubation chamber within entire incubation system 100 and thereby achieves a rapid equalization of the environmental conditions in all three incubation chambers 10, 20 and 30.

Incubation system 100 comprises a plurality of sensors 42 that is configured to determine at least one parameter of temperature, humidity, and gas concentration. The determination can be, in particular, time-resolved. One of these sensors 42 is disposed in first incubation chamber 10 and second incubation chamber 20 for recording the environmental conditions and outputting a measured value or a corresponding measurement signal. A third sensor 42 is disposed on sample carrier holder 15 and thereby in the immediate vicinity of a sample arranged therein. This sensor is therefore able to record the conditions in the vicinity of the sample. In particular, this sensor 42 is able to determine the parameters of the nutrient medium provided by sample supply device 40 so that measurement data from this sensor can be used to control sample supply device 40 in a closed-loop manner.

The measured values or measurement signals from sensors 42 are transmitted to a sensor input of a control device 50, for example, via an electrical connection. In this case, this electrical connection is an electrical feedthrough through the outer wall of the incubation chambers. Alternatively, the electrical connection can also be formed at an interface of the incubation chambers. It is also conceivable that the transmission is established by wireless connection, for example, via radio, Bluetooth, or wireless LAN. Control device 50 receives as further input a target value for the parameter to be controlled in a closed-loop manner. This parameter can be, for example, entered into control device 50 by a user via a user interface.

The values received from the sensor input are transferred to the processor of closed-loop control device 50. The processor is configured to calculate an error signal from the values received and the target value that corresponds to the deviation between of value measured by the sensor from the target value. This error signal serves as a closed-loop control signal. Depending on the parameter to be controlled in a closed-loop manner, the closed-loop control signal is transmitted from the closed-loop control signal output to incubation chamber supply device 41 and/or sample supply device 40. This closed-loop control signal means that the supply devices mentioned adapt their operation in such that the parameter to be controlled in a closed-loop manner is adapted to the target value. For example, if the temperature in incubation system 100 is too high, a heating device of incubation chamber supply device 41 can be ramped down. The closed-loop control process described is therefore based on a feedback-based control loop and can be carried out continuously.

Closed-loop control device 50 is shown as an external device, but it can also be present within incubation system 100. It is also possible that chamber supply device 41 and/or sample supply device 40 have components of closed-loop control device 50 implemented. In this case, the measured values from sensors 42 would be transmitted directly to chamber supply device 41 and/or sample supply device 40 and used there to control the respective devices in a closed-loop manner. The above explanations regarding closed-loop control device 50 also apply in this case.

The embodiments described are not to be construed to be restrictive. For example, the combinations of features are not tied to a specific number of incubation chambers or specific supply devices of incubation system 100.

1. Incubation device, comprising:
a first incubation chamber,
wherein the following are arranged in said first incubation chamber:
a sample carrier holder, and
an examination device for examining a sample which is arranged in said sample carrier holder,
wherein said first incubation chamber comprises a connection opening for connecting to a second incubation chamber and an inlet opening for introducing said sample into said first incubation chamber,
wherein a connection device is arranged at said connection opening, and
wherein said connection device can be connected to a connection device which is arranged at a connection opening of said second incubation chamber.

2. Incubation device according to claim 1, wherein said connection device of said first incubation chamber comprises a component of a screw connection, a magnetic connection with a positioning mechanism, a bayonet lock and/or a hook-in connection, for example, with a toggle latch.

3. Incubation device according to claim 1, furthermore comprising an interface in an outer wall of said first incubation chamber,
wherein said interface disposes of a port for an electrical, gas-conveying, and/or liquid-conveying connection.

4. Incubation device according to claim 3, wherein said interface comprises a plug connection.

5. Incubation device according to claim 1, wherein said first incubation chamber has a second connection opening at which a second connection device is arranged.

6. Modular incubation system, comprising:
Incubation device according to claim 1, and
a second incubation chamber with a connection opening,
wherein a connection device is arranged at said connection opening of said second incubation chamber,
wherein said connection device of said first incubation chamber is connected to said connection device of said second incubation chamber.

7. Incubation system according to claim 6, furthermore comprising a sample supply device for providing a gas or a liquid to said sample that is arranged in said sample carrier holder.

8. Incubation system according to claim 6, wherein an incubation device is provided comprising an interface in an outer wall of said first incubation chamber, wherein said interface disposes of a port for an electrical, gas-conveying, and/or liquid-conveying connection,
wherein an interface for connecting a gas-conveying and/or liquid-conveying connection is arranged in an outer wall of said second incubation chamber, and
wherein said interface of said first incubation chamber is connected to said interface of said second incubation chamber so that a closed gas-conveying and/or liquid-conveying connection is given.

9. Incubation system according to claim 6, furthermore comprising a heating element which is configured to adjust a temperature in said first incubation chamber and/or said second incubation chamber, and/or
a chamber supply device that is configured to adjust a humidity and/or gas concentration in said first incubation chamber and/or said second incubation chamber.

10. Incubation system according to claim 9, wherein said heating element is configured to adjust the temperature in said first incubation chamber and in said second incubation chamber independently of one another, and/or
wherein said chamber supply device is configured to adjust the air humidity and/or the gas concentration in said first incubation chamber and in said second incubation chamber independently of one another.

11. Incubation system according to claim 6, furthermore comprising an air diffuser, for example, a fan, for exchanging a temperature, gas concentration, and/or humidity between said first incubation chamber and said second incubation chamber.

12. Incubation system according to claim 6, wherein said first incubation chamber and/or said second incubation chamber are/is thermally insulated from an environment.

13. Incubation system according to claim 6, furthermore comprising:
a temperature sensor, air humidity sensor, and/or gas concentration sensor for determining at least one parameter of temperature, air humidity, and/or gas concentration in said first incubation chamber and/or said second incubation chamber.

14. Incubation system according to claim 13, furthermore comprising a closed-loop control device which is configured to control said parameter in a closed-loop manner based on an associated measured value from said sensor or said sensors.

15. Incubation system according to claim 6, furthermore comprising a third incubation chamber,
wherein said third incubation chamber comprises a connection opening with a connection device arranged thereat,
wherein said first incubation chamber or said second incubation chamber comprises a second connection opening with a connection device arranged thereat, and
wherein said connection device of said third incubation chamber is connected to said second connection device of said first incubation chamber or to said second connection device of said second incubation chamber.

16. Incubation device according to claim 4 wherein the plug connection has a sealing element.

17. Incubation device according to claim 7, wherein said sample supply device comprises a pump with a liquid reservoir.
